(19) United States
(12) Patent Application Publication
(10) Pub. No.: US 2004/0014691 A1
(43) Pub. Date: Jan. 22, 2004

(54) 9-Oxime macrolide antibacterials

(76) Inventors: Xenia Beebe Searle, Grayslake, IL (US); Stevan Djuric, Libertyville, IL (US); Zhenkun Ma, Dallas, TX (US); Fan Yang, Watertown, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/420,391

(22) Filed: Apr. 22, 2003

Related U.S. Application Data
(60) Provisional application No. 60/375,361, filed on Apr. 25, 2002.

(51) Int. Cl. C07H 17/08; A61K 31/7048

(52) U.S. Cl. 514/29; 536/7.4

(57) ABSTRACT

Antibacterial compounds having formula (I)

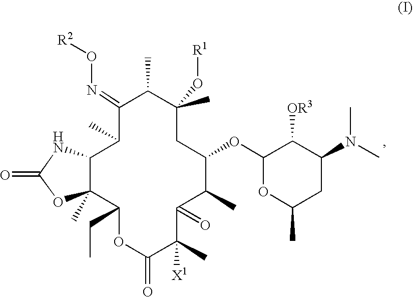

and salts, prodrugs, and salts of prodrugs thereof, processes for making the compounds and intermediates used in the processes, compositions containing the compounds, and methods for prophylaxis or treatment of bacterial infections using the compounds are disclosed.

This application claims the benefit of co-pending U.S. Provisional Application Ser. No. 60/375,361, filed Apr. 25, 2002, the specification of which is hereby incorporated into this application by reference.

### TECHNICAL FIELD

This invention is directed to compounds which are useful as antibacterials, processes for making the compounds and intermediates used in the processes, compositions containing the compounds, and methods for prophylaxis or treatment of bacterial infections using the compounds.

### BACKGROUND OF THE INVENTION

Because the effectiveness of many drugs currently available for prophylaxis or treatment of bacterial infections is being compromised by the emergence of drug-resistant bacteria, novel antibacterials would be beneficial for their therapeutic value and their contribution to the antibacterial arts.

### SUMMARY OF THE INVENTION

A first embodiment of this invention, therefore, is directed to compounds which are useful as antibacterials, and salts, prodrugs, and salts of prodrugs thereof, the compounds having formula (I)

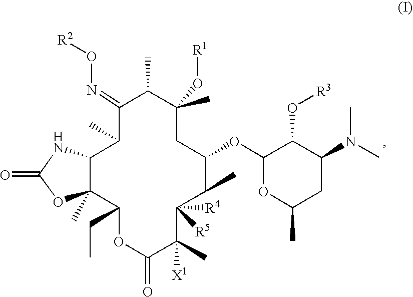

in which

R¹ is alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, and heterocyclyl, —(CH₂)alkenyl substituted with one or two or three substituents independently selected from the group consisting of cycloalkyl, halo, aryl, heteroaryl, and heterocyclyl, or —(CH₂)alkynyl substituted with one or two or three substituents independently selected from the group consisting of cycloalkyl, halo, aryl, heteroaryl, and heterocyclyl;

R² is hydrogen, alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, or —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;

R³ is hydrogen or R^{P}, in which R^{P} is a hydroxyl protecting moiety;

one of R⁴ and R⁵ is hydrogen and the other is —OH; or R⁴ and R⁵ together are ═O;

R⁶ is alkyl interrupted with one or two or three moieties independently selected from the group consisting of —O—, —NH—, —N(alkyl)- , —S—, —S(O)—, and —SO₂—, or alkyl interrupted with one or two or three moieties independently selected from the group consisting of —O—, —NH—, —N(alkyl)- , —S—, —S(O)—, and —SO₂— and substituted with one or two or three substituents independently selected from the group consisting of cycloalkyl, halo, aryl, heteroaryl, heterocyclyl, —OH, ═O, —OR⁷, —NH₂, —NHR⁸, and —NR⁸R⁹;

R⁷ is alkyl, heteroalkyl, aryl, heteroaryl, heterocyclyl, or alkyl substituted with one substituent selected from the group consisting of aryl, heteroaryl, and heterocyclyl;

R⁸ and R⁹ are independently alkyl, cycloalkyl, heteroalkyl, aryl, heteroaryl, heterocyclyl, or alkyl substituted with one substituent selected from the group consisting of aryl, heteroaryl, and heterocyclyl; or

R⁸ and R⁹ together are C₃-C₇-alkylene or C₄-C₇-alkylene interrupted with one moiety selected from the group consisting of an —O—, —NH—, —N(alkyl)-, —S—, —C(O)—, —S(O)—, and —SO₂—; and

X¹ is hydrogen or fluoro.

A second embodiment of this invention is directed to a process for making compounds having formula (I)-d

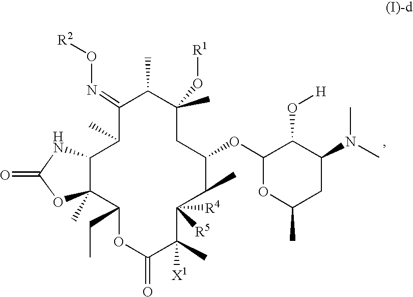

and salts, prodrugs, and salts of prodrugs thereof, in which

R¹ is alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, and heterocyclyl, —(CH₂)alkenyl substituted with one or two or three substituents independently selected from the group consisting of cycloalkyl, halo, aryl, heteroaryl, and heterocyclyl, or —(CH₂)alkynyl substituted with one or two or three substituents independently selected from the group consisting of cycloalkyl, halo, aryl, heteroaryl, and heterocyclyl;

R² is hydrogen, alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, heterocyclyl —OH, —OR⁷, ═O, —NH₂, —NHR⁸, and —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, or —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;

R⁴ and R⁵ taken together are ═O;

R⁶ is alkyl interrupted with one or two or three moieties independently selected from the group consisting of —O—, —NH—, —N(alkyl)-, —S—, —S(O)—, and —SO₂— or alkyl interrupted with one or two or three moieties independently selected from the group consisting of —O—, —NH—, —N(alkyl)-, —S—, —S(O)—, and —SO₂— and substituted with one or two or three substituents independently selected from the group consisting of cycloalkyl, halo, aryl, heteroaryl, heterocyclyl —OH, ═O, —OR⁷, —NH₂, —NHR⁸, and —NR⁸R⁹;

R⁷ is alkyl, heteroalkyl, aryl, heteroaryl, heterocyclyl, or alkyl substituted with one substituent selected from the group consisting of aryl, heteroaryl, and heterocyclyl;

R⁸ and R⁹ are independently alkyl, cycloalkyl, heteroalkyl, aryl, heteroaryl, heterocyclyl, or alkyl substituted with one substituent selected from the group consisting of aryl, heteroaryl, and heterocyclyl; or

R⁸ and R⁹ together are C₃-C₇-alkylene or C₄-C₇-alkylene interrupted with one moiety selected from the group consisting of —O—, —NH—, —N(alkyl)-, —S—, —C(O)—, —S(O)—, and —SO₂—; and

X¹ is hydrogen,

the process comprising the steps of:

(a) reacting compounds having formula (1)

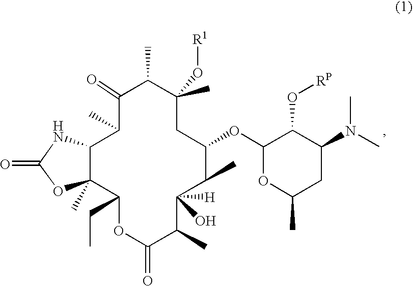

a preferred embodiment of which is

(2S,3R,4S,6R)-4-(dimethylamino)-2-(((3aS,4R,7R,8S,9S, 10R,11S,13R,15R,15aR) -4-ethyl-11-(prop-2-ynyloxy)-8-hydroxy-3a,7,9,11,13,15-hexamethyl-2,6,14-trioxotetradecahydro-2H-oxacyclotetradecino[4,3-d][1,3]oxazol-10-yl)oxy)-6-methyltetrahydro-2H-pyran-3-yl benzoate,

and

compounds having formula (2)

H₂N—OR² (2),

or salts thereof,

a preferred embodiment of which is methoxylamine hydrochloride,

with or without an additive, at a pressure between about 1.2 and 5 atm, to provide compounds having formula (I)-a

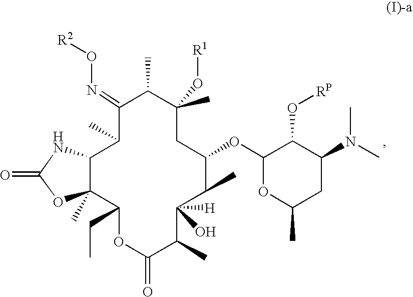

a preferred embodiment of which is

(2S,3R,4S,6R)-4-(dimethylamino)-2-(((3aS,4R,7R,8S,9S, 10R,11S,13R,14E,15R,15aR)-4-ethyl-11-(prop-2-ynyloxy)-8-hydroxy-14-(methoxyimino)-3a,7,9,11,13,15-hexamethyl-2,6-dioxotetradecahydro-2H-oxacyclotetradecino[4,3-d][1,3]oxazol-10-yl)oxy)-6-methyltetrahydro-2H-pyran-3-yl benzoate;

(b) reacting the product of step (a), an oxidant, with or without a first base, to provide a compound having formula (I)-b

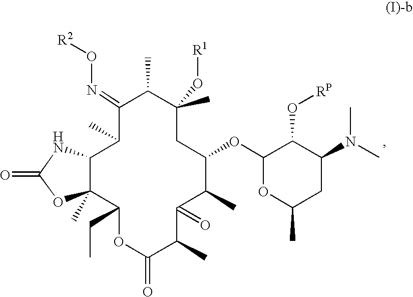

a preferred embodiment of which is

(2S,3R,4S,6R)-4-(dimethylamino)-2-(((3aS,4R,7R,8S,9R, 10R,11S,13R,14E,15R,15aR) -4-ethyl-11-(prop-2-ynyloxy)-14-(methoxyimino)-3a,7,9,11,13,15-hexamethyl-2,6,8-trioxotetradecahydro-2H-oxacyclotetradecino[4,3-d][1,3]oxazol-10-yl)oxy)-6-methyltetrahydro-2H-pyran-3-yl benzoate;

and

(c) reacting the product of step (b) and methanol.

A third embodiment of this invention is directed to intermediates which are used in the second embodiment.

A fourth embodiment of this invention is directed to a process for making compounds having formula (I)-c

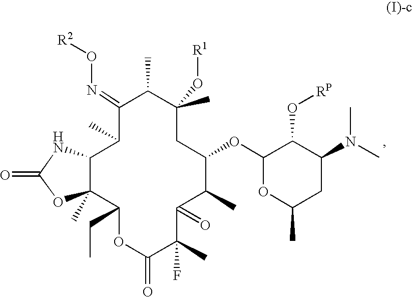

and salts, prodrugs, and salts of prodrugs thereof,

in which

R¹ is alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, and heterocyclyl, —(CH₂)alkenyl substituted with one or two or three substituents independently selected from the group consisting of cycloalkyl, halo, aryl, heteroaryl, and heterocyclyl, or —(CH₂)alkynyl substituted with one or two or three substituents independently selected from the group consisting of cycloalkyl, halo, aryl, heteroaryl, and heterocyclyl;

R² is hydrogen, alkyl, —(CH₂)alkenyl, —(CH₂)alkynyl, —CH₂R⁶, alkyl substituted with one, two, or three substituents independently selected from the group consisting of cycloalkyl, halo, aryl, heteroaryl, heterocyclyl, —OH, —OR⁷, ═O, —NH₂, —NHR⁸, or —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, and —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;

R^{P} is acetyl or benzoyl;

R⁶ is alkyl interrupted with one or two or three moieties independently selected from the group consisting of —O—, —NH—, —N(alkyl)-, —S—, —S(O)—, and —SO₂— or alkyl independently interrupted with one or two or three moieties independently selected from the group consisting of —O—, —NH—, —N(alkyl)-, —S—, —S(O)—, and —SO₂— and substituted with one or two or three substituents independently selected from the group consisting of cycloalkyl, halo, aryl, heteroaryl, heterocyclyl —OH, ═O, —OR⁷, —NH₂, —NHR⁸, and —NR⁸R⁹;

R⁷ is alkyl, heteroalkyl, aryl, heteroaryl, heterocyclyl, or alkyl substituted with one substituent selected from the group consisting of aryl, heteroaryl, and heterocyclyl;

R⁸ and R⁹ are independently alkyl, cycloalkyl, heteroalkyl, aryl, heteroaryl, heterocyclyl, or alkyl substituted with one substituent independently selected from the group consisting of aryl, heteroaryl, and heterocyclyl; or

R⁸ and R⁹ together are C₃-C₇-alkylene or C₄-C₇-alkylene interrupted with one moiety selected from the group consisting of —O—, —NH—, —N(alkyl)-, —S—, —C(O)—, —S(O)—, and —SO₂—,

the process comprising the step of:

(a) reacting compounds having formula (I)-b

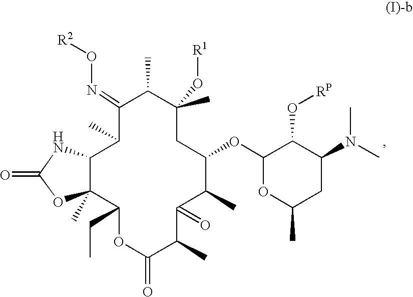

a preferred embodiment of which is

(2S,3R,4S,6R)-4-(dimethylamino)-2-(((3aS,4R,7R,8S,9R, 10R,11S,13R,14E,15R,15aR)-4-ethyl-11-(prop-2-ynyloxy)-14-(methoxyimino)-3a,7,9,11,13,15-hexamethyl-2,6,8-trioxotetradecahydro-2H-oxacyclotetradecino[4,3-d][1,3]oxazol-10-yl)oxy)-6-methyltetrahydro-2H-pyran-3-yl benzoate,

and a fluoridating agent, with or without a second base.

A fifth embodiment of this invention is directed to intermediates which are used in the fourth embodiment.

A sixth embodiment of this invention is directed to compositions which are useful for the treatment and prophylaxis of bacterial infections in a fish or a mammal, the compositions comprising a therapeutically effective amount of one or more of the compounds of the first embodiment and an excipient.

A seventh embodiment of this invention is directed to methods for prophylaxis or treatment of bacterial infections in a fish or a mammal, the method comprising administering to the fish or the mammal a therapeutically effective amount of one or more of the compounds of the first embodiment.

### DETAILED DESCRIPTION OF THE INVENTION

Compounds of this invention, also referred to as “the compounds,” comprise both fixed and variable moieties, which variable moieties are identified by a capital letter and accompanying numerical or alphabetical superscript, and for which the following terms have the meanings indicated.

“Alkenyl” means monovalent, straight-chain and branched-chain hydrocarbon moieties, having two to eight carbon atoms and at least one carbon-carbon double bond.

Alkenyl moieties include but-1,3-dienyl, butenyl, but-2-enyl, ethenyl, 1-ethylhexen-2-yl, hex-3-enyl, 1-methylbutenyl, 2-methylbutenyl, 1-methylbut-2-enyl, 1-methylbut-1,3-dienyl, pentenyl, pent-2-enyl, and pent-3-enyl, propenyl.

“Alkyl” means monovalent, saturated, straight-chain and branched-chain hydrocarbon moieties, having one to six carbon atoms.

Alkyl moieties include butyl, 1,1,-dimethylethyl, 1,1-dimethylpropyl, 1,2-dimethylpropyl, ethyl, 1-ethylpropyl, 2-ethylpropyl, hexyl, methyl, 2-methylpropyl, 3-methylbutyl, 1-methylpentyl, 2-methylpent-3-yl, and pentyl.

“Alkylene” means divalent, saturated, straight-chain and branched-chain hydrocarbon moieties, having one to eight carbon atoms.

Alkylene moieties include butylene, 1,1,-dimethylethylene, 1,1-dimethylpropylene, 1,2-dimethylpropylene, ethylene, 1-ethylpropylene, 2-ethylpropylene, hexylene, methylene, 2-methylpropylene, 3-methylbutylene, 1-methylpentylene, 2-methylpent-3-ylene, and pentylene.

“Alkynyl” means monovalent, straight-chain and branched-chain hydrocarbon moieties, having two to six carbon atoms and at least one carbon-carbon triple bond.

Alkynyl moieties include ethynyl (acetylenyl), pentynyl, pent-2-ynyl, pent-3-ynyl, pent-4-ynyl, 1-methylbut-2-ynyl, 2-methylbut-3-ynyl, hexynyl, hex-2-ynyl, hex-3-ynyl, hex-4-ynyl, 1-methyl-pent-2-ynyl, 1-methylenepent-3-ynyl, 1-methyl-pent-2,4-diynyl, and prop-2-ynyl (propargyl).

“Aryl” means monovalent, unsubstituted and substituted phenyl moieties, attached through a carbon atom, and unfused or fused with another phenyl moiety or a cycloalkyl, cycloalkenyl, heteroaryl, heterocyclyl, naphthyl, or saturated part of an indanyl moiety.

Phenyl moieties fused with phenyl, naphthyl, or the saturated part of an indanyl moieties are unsubstituted and substituted naphthyl, anthracen-(1- to 4-)yl, or fluoren-(1- to 4-)yl, respectively.

Phenyl moieties fused with cycloalkyl moieties are unsubstituted and substituted indan-(4- to 7-)yl and 1,2,3,4-tetrahydronaphth-(5- to 8-)yl.

Phenyl moieties fused with cycloalkenyl moieties are unsubstituted and substituted inden-(4- to 7-)yl, 1,2-dihydronaphth-(5- to 8-)yl and 1,2-dihydronaphth-(5- to 8-)yl.

Phenyl moieties fused with heteroaryl moieties include unsubstituted and substituted benzimidazol-(4- to 7-)yl, 1-benzofuran-(4- to 7-)yl, 1,2-benzisothiazol-(4- to 7-)yl, benzthiazol-(4- to 7-)yl, 1-benzothiophen-(4- to 7-)yl, cinnolin-(5- to 8-)yl, indol-(4- to 7-)yl, isoquinolin-(5- to 8-)yl, phthalazin-(5- to 8-)yl, quinazolin-(5- to 8-)yl, quinolin-(5- to 8-)yl, and quinoxalin-(5- to 8-)yl.

Phenyl moieties fused with heterocyclyl moieties include unsubstituted and substituted 1,3-benzodiox-(4- to 7-)yl, 1,4-benzodiox-(5- to 8-)yl, 1,3-dihydro-2-benzofuran-(4- to 7-)yl, 2,3-dihydro-1-benzofuran-(4- to 7-)yl, 1,3-dihydro-2-benzothiophen-(4- to 7-)yl, 2,3-dihydro-1-benzothiophen-(4- to 7-)yl, and indolin-(4- to 7-)yl.

“Cycloalkyl” means monovalent, unsubstituted and substituted, saturated cyclic hydrocarbon moieties, having three to six carbon atoms.

Cycloalkyl moieties are unsubstituted and substituted cyclopropyl, cyclobutyl, cyclopentyl, and cyclohexyl.

“Cycloalkenyl” means means monovalent, unsubstituted and substituted, cyclic hydrocarbon moieties having four to six carbon atoms and at least one carbon-carbon double bond.

Cycloalkenyl moieties are unsubstituted and substituted 1,3-cyclohexadienyl, 1,4-cyclohexadienyl, cyclohexenyl, cyclopentadienyl, and cyclopentenyl.

“Halo” means fluoro (—F), chloro (—Cl), bromo (—Br), and iodo (—I) moieties.

“Heteroaryl” means monovalent, aromatic, unsubstituted and substituted five-membered ring moieties having two double bonds and (a) one oxygen or one sulfur atom, (b) one, two, three, or four nitrogen atoms, or (c) one or two nitrogen atoms and one oxygen or one sulfur atom and the remaining atoms are carbon atoms, each of which is attached through a carbon atom or a nitrogen atom; and monovalent six-membered ring moieties having three double bonds and one, two, or three nitrogen atoms and the remaining atoms are carbon atoms, attached through a carbon atom; in which the foregoing heteroaryl moieties are unfused or fused with another heteroaryl moiety or an aryl moiety.

Five-membered heteroaryl moieties are unsubstituted and substituted furanyl, imidazolyl, isothiazolyl, isoxazolyl, 1,2,3-oxadiazolyl, oxazolyl, pyrazolyl, pyrrolyl, tetrazolyl, 1,3,4-thiadiazolyl, thiazolyl, thiophenyl (thienyl), 2H-tetraäzolyl, and 1,2,3-triazolyl.

Five-membered heteroaryl moieties fused with aryl moieties include unsubstituted and substituted benzimidazol-(1- or 2-)yl, 1-benzofuran-(2- to 3-)yl, 1,2-benzisothiazol-3-yl, benzthiazol-2-yl, 1-benzothiophen-(2- to 3-)yl, cinnolin-(3- or 4-)yl, indol-(1- to 3-)yl, isoquinolin-(1-, 3-, or 4-)yl, phthalazin-(1- or 4-)yl, quinazolin-(2- or 4-)yl, quinolin-(2- to 4-)yl, and quinoxalin-(2- or 3-)yl.

Five-membered heteroaryl moieties fused with other five-membered heteroaryl moieties include unsubstituted and substituted [1,3]thiazolo[4,5-d][1,3]oxazolyl, [1,3]thiazolo[4,5-d][1,3]thiazolyl, thieno[3,2-d][1,3]oxazolyl, thieno[3,2-d][1,3]thiazolyl, and thieno[2,3-b]thiophenyl.

Five-membered heteroaryl moieties fused with six-membered heteroaryl moieties include unsubstituted and substituted furo[2,3-b]pyridin-(2- or 3-)yl, 3H-imidazo[4,5-b]pyridin-(2- or 3-)yl, [1,3]thiazolo[4,5-b]pyrazin-2-yl, [1,3]thiazolo[4,5-b]pyridin-2-yl, and thieno[2,3-b]pyridin-(2- or 3-)yl.

Six-membered heteroaryl moieties are unsubstituted and substituted pyrazinyl, pyridazinyl, pyridyl, pyrimidinyl, and 1,3,5-triazinyl.

Six-membered heteroaryl moieties fused with aryl moieties include unsubstituted and substituted cinnolin-(3- or 4-)yl, isoquinolin-(1-, 3-, or 4-)yl, phthalazin-(1- or 4-)yl, quinazolin-(2- or 4-)yl, quinolin-(2- to 4-)yl, and quinoxalin-(2- or 3-)yl.

Six-membered heteroaryl moieties fused with five-membered heteroaryl moieties include unsubstituted and substituted furo[2,3-b]pyridin-(4- to 6-)yl, 3H-imidazo[4,5-b]pyridin-(5- to 7-)yl, [1,3]thiazolo[4,5-b]pyrazin-(5- or 6-)yl, [1,3]thiazolo[4,5-b]pyridin-(5- to 7-)yl, and thieno[2,3-b]pyridin-(4- to 6-)yl.

Six-membered heteroaryl moieties fused with other six-membered heteroaryl moieties include unsubstituted and substituted 1,5-naphthyridinyl, 1,7-naphthyridinyl, 1,8-naphthyridinyl, pteridinyl, pyridazino[4,5-d]pyridazinyl, pyrido[2,3-d]pyridazinyl, and pyrido[3,4-d]pyridazinyl.

“Heterocyclyl” means (a) monovalent, non-aromatic, unsubstituted and substituted four-membered ring moieties having one nitrogen, oxygen, or sulfur atom and the remaining atoms are carbon atoms, zero double bonds, attached through a carbon atom or a nitrogen atom, (b) monovalent, non-aromatic, unsubstituted and substituted five-membered ring moieties having one or two nitrogen, oxygen, or sulfur atoms and the remaining atoms are carbon atoms, and zero or one double bonds, attached through a carbon atom or a nitrogen atom, and (c) monovalent, non-aromatic, unsubstituted and substituted six-membered ring moieties having one, two, or three nitrogen, oxygen, or sulfur atoms and the remaining atoms are carbon atoms, and zero, one, or two double bonds, attached through a carbon atom or a nitrogen atom.

Four-membered heterocyclyl moieties are unsubstituted and substituted oxetane, thietane, and azetidine.

Five-membered heterocyclyl moieties include unsubstituted and substituted 1,4-dioxanyl, 1,3-dioxolanyl, imidazolidinyl, 2-imidazolinyl, 4,5-dihydroisoxazolyl, pyrazolidinyl, 2-pyrazolinyl, pyrrolidinyl, 2-pyrrolinyl, 3-pyrrolinyl, and 2H-pyrrolyl.

Six-membered heterocyclyl moieties include unsubstituted and substituted 1,3-dithianyl, 1,4-dithianyl, morpholinyl, piperidinyl, piperazinyl, pyranyl, 2H-pyranyl, 4H-pyranyl, and thiomorpholinyl.

Substituted aryl and heteroaryl moieties are those moieties substituted with one, two, three, four, or five substituents independently selected from the group consisting of alkyl, alkenyl, alkynyl, cycloalkyl, halo, —CN, —OH, —SH, —NH₂, —NO₂, —CF₃, —CH₂CF₃, —CF₂CF₃, —OCF₃, —OCH₂CF₃, —OCF₂CF₃, —OR³⁰, —SR³⁰, —S(O)(alkyl), —SO₂(alkyl), —C(O)H, —C(O)(alkyl), —C(O)OH, —C(O)O(alkyl), —NH(alkyl), —N(alkyl)₂, —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), —NHC(O)N(alkyl)₂, —SO₂NH₂, —SO₂NH(alkyl), —SO₂N(alkyl)₂, and R⁴⁰, in which R³⁰ is alkyl or alkyl substituted with one substituent selected from the group consisting of halo, —O(alkyl), and —S(alkyl), and R⁴⁰ is furyl, imidazolyl, indazolidinyl, isoquinolyl, isothiazolyl, isoxazolyl, morpholinyl, naphthyl, naphthyridyl, 1,2,3-oxadiazolyl, oxazolyl, phenyl, piperidinyl, piperazinyl, pyrazinyl, pyrazolyl, pyridyl, pyrimidinyl, pyrrolidinyl, pyrrolyl, quinazolyl, quinolyl, quinoxalyl, tetraäzolyl, 1,2,3-thiadiazolyl, 1,3,4-thiadiazolyl, thiazolyl, thienyl, 1,2,3-triazolyl, or thiomorpholinyl, in which each R⁴⁰ moiety is unsubstituted or substituted with one or two or three substituents independently selected from the group consisting of alkyl, alkenyl, alkynyl, cycloalkyl, halo, ═O, —CN, —OH, —SH, —NO₂, —CF₃, —CH₂CF₃, —CF₂CF₃, —OCF₃, —OCH₂CF₃, —OCF₂CF₃, —O(alkyl), —S(alkyl), —S(O)(alkyl), —SO₂(alkyl), —C(O)H, —C(O)(alkyl), —C(O)OH, —C(O)O(alky(l), —NH₂, —NH(alkyl), —N(alkyl)₂, —C(O)NH₂, —C(O)NH(alkyl), —C(O)N(alkyl)₂, —OC(O) (alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), —NHC(O)N(alkyl)₂, —SO₂NH₂, —SO₂NH(alkyl), and —SO₂N(alkyl)₂.

Substituted cycloalkyl, cycloalkenyl, and heterocyclyl moieties are those moieties substituted with one, two, or three substituents independently selected from the group consisting of alkyl, phenyl, halo, —CN, —OH, —NH₂, —CF₃, —OR³⁰, —SR³⁰, —S(O)(alkyl), —SO₂(alkyl), —C(O)H, —C(O)(alkyl), —C(O)OH, —C(O)O(alkyl), —NH(alkyl), —N(alkyl)₂, —C(O)NH₂, —C(O)NH(alkyl), and —C(O)N(alkyl)₂, in which the phenyl is unsubstituted or substituted with one, two, or three substituents independently selected from the group consisting of halo, —CN, —OH, —NH₂, and —CF₃.

“Hydroxyl protecting moiety” means selectively introducible and removable moieties which protect —OH moieties against undesirable side reactions. Hydroxyl protecting moieties include 4-nitrobenzyloxycarbonyl, 4-bromobenzyloxycarbonyl, 4-methoxybenzyloxycarbonyl, 3,4-dimethoxybenzyloxycarbonyl, tert-butoxycarbonyl, diphenylmethoxycarbonyl, 2,2,2-trichloroethoxycarbonyl, 2,2,2-tribromoethoxycarbonyl, 2-(trimethylsilyl)ethoxycarbonyl, 2-(phenylsulfonyl)ethoxycarbonyl, allyloxycarbonyl, acetyl, chloroacetyl, dichloroacetyl, trichloroacetyl, trifluoroacetyl, methoxyacetyl, phenoxyacetyl, pivaloyl, propionyl, 2-methylpropionyl, benzoyl, tert-butyl, 2,2,2-trichloroethyl, 2-trimethylsilylethyl, 1,1-dimethyl-2-propenyl, 3-methyl-3-butenyl, para-methoxybenzyl, 3,4-dimethoxybenzyl, diphenylmethyl, triphenylmethyl, tetrahydrofuryl, benzyloxymethyl, 2-methoxyethoxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, diethylisopropylsilyl, tert-butyldimethylsilyl, tert-butyldiphenylsilyl, diphenylmethylsilyl, and tert-butylmethoxyphenylsilyl.

These variable moieties may combine to provide an eighth embodiment of this invention, which embodiment is directed to compounds having formula (I) and pharmaceutically acceptable salts, prodrugs, and salts of prodrugs thereof, in which

R¹ is —(CH₂)alkenyl, —(CH₂)alkynyl, —(CH₂)alkenyl substituted with one substituent selected from the group consisting of cycloalkyl, halo, aryl, heteroaryl, and heterocyclyl, or —(CH₂)alkynyl substituted with one substituent selected from the group consisting of cycloalkyl, halo, aryl, heteroaryl, and heterocyclyl;

R² is hydrogen, alkyl, —(CH₂)alkenyl, —(CH₂)alkynyl, alkyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, or —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;

R³ is hydrogen or R^{P}, in which R^{P} is a hydroxyl protecting moiety;

one of R⁴ and R⁵ is hydrogen and the other is —OH; or R⁴ and R⁵ together are ═O;

R⁷ is alkyl, heteroalkyl, aryl, heteroaryl, heterocyclyl, or alkyl substituted with one substituent selected from the group consisting of aryl, heteroaryl, and heterocyclyl;

R⁸ and R⁹ are independently alkyl, cycloalkyl, heteroalkyl, aryl, heteroaryl, heterocyclyl, or alkyl substituted with one substituent selected from the group consisting of aryl, heteroaryl, and heterocyclyl; and

X¹ is hydrogen or fluoro;

compounds having formula (I), and pharmaceutically acceptable salts, prodrugs, and salts of prodrugs thereof, in which

R¹ is —(CH₂)alkynyl or —(CH₂)alkynyl substituted with one substituent selected from the group consisting of cycloalkyl, halo, aryl, heteroaryl, and heterocyclyl;

R² is hydrogen, alkyl, —(CH₂)alkenyl, —(CH₂)alkynyl, alkyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, or —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;

R³ is hydrogen or R^{P}, in which R^{P} is a hydroxyl protecting moiety;

one of R⁴ and R⁵ is hydrogen and the other is —OH; or R⁴ and R⁵ together are ═O;

R⁷ is alkyl, heteroalkyl, aryl, heteroaryl, heterocyclyl, or alkyl substituted with one substituent selected from the group consisting of aryl, heteroaryl, and heterocyclyl;

R⁸ and R⁹ are independently alkyl, cycloalkyl, heteroalkyl, aryl, heteroaryl, heterocyclyl, or alkyl substituted with one substituent selected from the group consisting of aryl, heteroaryl, and heterocyclyl; and

X¹ is hydrogen or fluoro;

compounds having formula (I), and pharmaceutically acceptable salts, prodrugs, and salts of prodrugs thereof, in which

R¹ is —(CH₂)alkynyl or —(CH₂)alkynyl substituted with one substituent selected from the group consisting of aryl and heteroaryl;

R² is hydrogen, alkyl, or alkyl substituted with one substituent selected from the group consisting of cycloalkyl, halo, aryl, heteroaryl, and heterocyclyl;

R³ is hydrogen or R^{P}, in which R^{P} is a hydroxyl protecting moiety;

one of R⁴ and R⁵ is hydrogen and the other is —OH; or R⁴ and R⁵ together are ═O; and

X¹ is hydrogen or fluoro;

compounds having formula (I), and pharmaceutically acceptable salts, prodrugs, and salts of prodrugs thereof, in which

R¹ is —(CH₂)alkynyl or —(CH₂)alkynyl substituted with one substituent selected from the group consisting of aryl and heteroaryl;

R² is hydrogen, alkyl, or alkyl substituted with phenyl;

R³ is hydrogen or R^{P}, in which R^{P} is a hydroxyl protecting moiety;

one of R⁴ and R⁵ is hydrogen and the other is —OH; or R⁴ and R⁵ together are ═O; and

X¹ is hydrogen or fluoro;

compounds having formula (I), and pharmaceutically acceptable salts, prodrugs, and salts of prodrugs thereof, in which R¹ is —(CH₂)alkynyl or —(CH₂)alkynyl substituted with one substituent selected from the group consisting of aryl and heteroaryl; R² is alkyl; R³ is hydrogen or R^{P}, in which R^{P} is a hydroxyl protecting moiety; one of R⁴ and R⁵ is hydrogen and the other is —OH; or R⁴ and R⁵ together are ═O; and X¹ is hydrogen or fluoro;

compounds having formula (I), and pharmaceutically acceptable salts, prodrugs, and salts of prodrugs thereof, in which

R¹ is —(CH₂)alkynyl or —(CH₂)alkynyl substituted with one substituent selected from the group consisting of quinoxalinyl, naphthyridinyl, quinolinyl, isoquinolinyl, thienyl, isoxazolyl, thiazolyl, pyridyl and pyrimidinyl, in which the thienyl, isoxazolyl, thiazolyl, pyridyl and pyrimidinyl are independently unsubstituted or substituted with one substituent selected from the group consisting of pyrimidinyl, pyridyl, thienyl, isoxazolyl, thiazolyl, 1H-tetraäzol-5-yl, 2H-tetraäzol-5-yl, 1-methyl-1H-tetraäzol-5-yl, and 2-methyl-2H-tetraäzol-5-yl;

R² is alkyl;

R³ is hydrogen or R^{P}, in which R^{P} is a hydroxyl protecting moiety;

one of R⁴ and R⁵ is hydrogen and the other is —OH; or R⁴ and R⁵ together are ═O; and

X¹ is hydrogen or fluoro;

compounds having formula (I), and pharmaceutically acceptable salts, prodrugs, and salts of prodrugs thereof, in which

R¹ is prop-2-ynyl, 3-(quinolin-3-yl)prop-2-ynyl, 3-(1,5-naphthyridin-3-yl)prop-2-ynyl, 3-(quinoxalin-6-yl)prop-2-ynyl, 3-(5-(2-methyl-2H-tetraäzol-5-yl)thien-2-yl)prop-2-ynyl, 3-(5-(pyridin-2-yl)thien-2-yl)prop-2-ynyl, 3-(2-(pyrimidin-2-yl)thien-5-yl)prop-2-ynyl, 3-(2-(2-methyl-2H-tetraäzol-5-yl)1,3-thiazol-5-yl)prop-2-ynyl, 3-(2-(2-methyl-2H-tetraäzol-5-yl)pyridin-6-yl)prop-2-ynyl, 3-(3-(pyridin-2-yl)isoxazol-5-yl)prop-2-ynyl, or 3-(3-(2-methyl-2H-tetraäzol-5-yl)isoxazol-5-yl)prop-2-ynyl;

R² is alkyl;

R³ is hydrogen or R^{P}, in which R^{P} is a hydroxyl protecting moiety;

one of R⁴ and R⁵ are hydrogen and the other is —OH; or R⁴ and R⁵ together are ═O; and

X¹ is hydrogen or fluoro; and

compounds having formula (I), and pharmaceutically acceptable salts, prodrugs, and salts of prodrugs thereof, in which

R¹ is —(CH₂)alkynyl or —(CH₂)alkynyl substituted with one substituent selected from the group consisting of aryl and heteroaryl;

R² is methyl;

R³ is hydrogen or R^{P}, in which R^{P} is a hydroxyl protecting moiety;

one of R⁴ and R⁵ is hydrogen and the other is —OH; or R⁴ and R⁵ together are ═O; and

X¹ is hydrogen or fluoro.

Specific examples of R¹ moieties are 3-(quinolin-3-yl)prop-2-ynyl, 3-(1,5-naphthyridin-3-yl)prop-2-ynyl, 3-(quinoxalin-6-yl)prop-2-ynyl, 3-(5-(2-methyl-2H-tetraäzol-5-yl)thien-2-yl)prop-2-ynyl, and 3-(5-(pyridin-2-yl)thien-2-yl)prop-2-ynyl.

A specific example of an R² moiety is methyl.

A specific example of an R³ moiety is hydrogen.

A specific example of R⁴ and R⁵ moieties is R⁴ and R⁵ taken together as ═O.

Specific examples of X¹ moieties are hydrogen and fluoro;

These specific moieties of the compounds may combine with the fixed moieties thereof to form a ninth embodiment of this invention, which embodiment is directed to compounds, and salts, prodrugs, and salts of prodrugs thereof, which are useful as antibacterials, the compounds having formula (I)

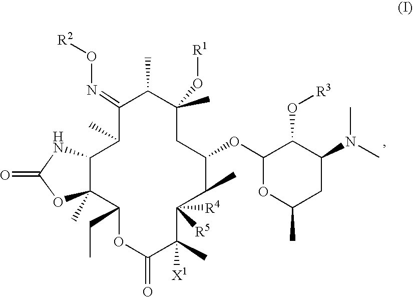

in which R¹ is —(CH₂)alkynyl substituted with one substituent selected from the group consisting of naphthyridinyl, quinolinyl, quinoxalinyl, and thienyl, in which the thienyl is substituted with one substituent selected from the group consisting of pyridyl and 2H-tetraäzolyl, in which the 2H-tetraäzolyl is substituted with alkyl; R² is alkyl; R³ is hydrogen; R⁴ and R⁵ taken together are ═O; and X¹ is hydrogen or fluoro;

a tenth embodiment of this invention, which embodiment is directed to compounds, and salts, prodrugs, and salts of thereof, having formula (I)

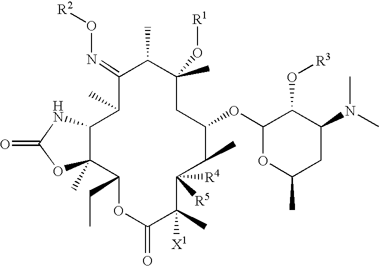

in which R¹ is —(CH₂)—C₂-alkynyl substituted with one substituent selected from the group consisting of naphthyridinyl, quinolinyl, quinoxalinyl, and thienyl, in which the thienyl is substituted with one substituent selected from the group consisting of pyridyl and 2H-tetraäzolyl, in which the 2H-tetraäolyl is substituted with C₁-alkyl; R² is C₁-alkyl; R³ is hydrogen; R⁴ and R⁵ taken together are ═O; and X¹ is hydrogen or fluoro;

compounds having formula (I), and salts, prodrugs, and salts of prodrugs thereof, in which R¹ is 3-(quinolin-3-yl)prop-2-ynyl, 3-(1,5-naphthyridin-7-yl)prop-2-ynyl, or 3-(quinoxalin-7-yl)prop-2-ynyl; R² is methyl; R³ is hydrogen; R⁴ and R⁵ taken together are ═O; and X¹ is hydrogen;

compounds having formula (I), and salts, prodrugs, and salts of prodrugs thereof, in which R¹ is 3-(quinolin-3-yl)prop-2-ynyl, 3-(1,5-naphthyridin-3-yl)prop-2-ynyl, or 3-(quinoxalin-6-yl)prop-2-ynyl; R² is methyl; R³ is hydrogen; R⁴ and R⁵ taken together are ═O; and X¹ is fluoro;

compounds having formula (I), and salts, prodrugs, and salts of prodrugs thereof, in which R¹ is 3-(5-(2-methyl-2H-tetraäzol-5-yl)thien-2-yl)prop-2-ynyl, or 3-(5-(pyridin-2-yl)thien-2-yl)prop-2-ynyl; R² is methyl; R³ is hydrogen; R⁴ and R⁵ taken together are ═O; and X¹ is hydrogen;

compounds having formula (I), and salts, prodrugs, and salts of prodrugs thereof, in which R¹ is 3-(5-(2-methyl-2H-tetraäzol-5-yl)thien-2-yl)prop-2-ynyl, or 3-(5-(pyridin-2-yl)thien-2-yl)prop-2-ynyl; R² is methyl; R³ is hydrogen; R⁴ and R⁵ taken together are ═O; and X¹ is fluoro; and

compounds, and salts, prodrugs, and salts of prodrugs thereof, which are

(3aS,4R,7R,9R,10R,11S,13R,14E,15R,15aR)-4-ethyl-14-(methoxyimino)-3a,7,9,11,13,15-hexamethyl-2,6,8-trioxo-11-((3-quinolin-3-ylprop-2-ynyl)oxy)tetradecahydro-2H-oxacyclotetradecino(4,3-d)(1,3)oxazol-10-yl 3,4,6-trideoxy-3-(dimethylamino)-beta-D-xylo-hexopyranoside;

(3aS,4R,7R,9R,10R,11S,13R,14E,15R,15aR)-4-ethyl-14-(methoxyimino)-3a,7,9,11,13,15-hexamethyl-11-((3-(1,5-naphthyridin-3-yl)prop-2-ynyl)oxy)-2,6,8-trioxotetradecahydro-2H-oxacyclotetradecino(4,3-d)(1,3)oxazol-10-yl 3,4,6-trideoxy-3-(dimethylamino)-β-D-xylo-hexopyranoside;

(3aS,4R,7R,9R,10R,11S,13R,14E,15R,15aR)-4-ethyl-14-(methoxyimino)-3a,7,9,11,13,15-hexamethyl-2,6,8-trioxo-11-((3-quinoxalin-6-ylprop-2-ynyl)oxy)tetradecahydro-2H-oxacyclotetradecino(4,3-d)(1,3)oxazol-10-yl 3,4,6-trideoxy-3-(dimethylamino)-β-D-xylo-hexopyranoside;

(3aS,4R,7R,9R,10R,11S,13R,14E,15R,15aR)-4-ethyl-14-(methoxyimino)-3a,7,9,11,13,15-hexamethyl-11-((3-(5-(2-methyl-2H-tetraäzol-5-yl)thien-2-yl)prop-2-ynyl)oxy)-2,6,8-trioxotetradecahydro-2H-oxacyclotetradecino(4,3-d)(1,3)-oxazol-10-yl 3,4,6-trideoxy-3-(dimethylamino)-β-D-xylohexopyranoside;

(3aS,4R,7R,9R,10R,11S,13R,14E,15R,15aR)-4-ethyl-14-(methoxyimino)-3a,7,9,11,13,15-hexamethyl-2,6,8-trioxo-11-((3-(5-pyridin-2-ylthien-2-yl)prop-2-ynyl)oxy)tetradecahydro-2H-oxacyclotetradecino(4,3-d)(1,3)oxazol-10-yl 3,4,6-trideoxy-3-(dimethylamino)-β-D-xylo-hexopyranoside;

(3aS,4R,7S,9R,10R,11S,13R,14E,15R,15aR)-4-ethyl-7-fluoro-14-(methoxyimino)-3a,7,9,11,13,15-hexamethyl-2,6,8-trioxo-11-((3-quinolin-3-ylprop-2-ynyl)oxy)tetradecahydro-2H-oxacyclotetradecino(4,3-d)(1,3) oxazol-10-yl 3,4,6-trideoxy-3-(dimethylamino)-β-D-xylo-hexopyranoside; and

(3aS,4R,7S,9R,10R,11S,13R,14E,15R,15aR)-4-ethyl-7-fluoro-14-(methoxyimino)-3a,7,9,11,13,15-hexamethyl-11-((3-(5-(2-methyl-2H-tetraäzol-5-yl)thien-2-yl)prop-2-ynyl)oxy)-2,6,8-trioxotetradecahydro-2H-oxacyclotetradecino-(4,3-d)(1,3)oxazol-10-yl 3,4,6-trideoxy-3-(dimethylamino)-β-D-xylo-hexopyranoside.

Compounds of this invention contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration which is present in the higher amount, preferably an excess of about 85%-90%, more preferably an excess of about 95%-99%, and still more preferably an excess greater than about 99%. Accordingly, this invention is meant to embrace all stereoisomers of the compounds including racemic mixtures, enantiomers, mixtures of enantiomers, diastereomers, and mixtures of diastereomers.

Individual stereoisomers of the compounds may be prepared by any one of a number of methods within the knowledge of the ordinarily skilled practioner. These methods include stereospecific synthesis, chromatographic separation of diastereomers, chromatographic resolution of enantiomers, enzymatic resolution, and conversion of enantiomers in an enantiomeric mixture to diastereomers and chromatographically separating the diastereomers and regeneration of the individual enantiomers.

Stereospecific synthesis involves the use of appropriate chiral starting materials and synthetic reactions which do not cause racemization or inversion of stereochemistry at the chiral centers.

Diastereomeric mixtures of compounds resulting from a synthetic reaction can be separated by chromatographic techniques which are well-known to the ordinarily skilled practioner.

Chromatographic resolution of enantiomers can be accomplished on chiral commercially available chromatography resins. In practice, the racemate is placed in solution and loaded onto the column containing a chiral stationary phase. The enantiomers are then separated by high performance liquid chromatography.

Enzymes, such as esterases, phosphatases and lipases, may be useful for resolution of derivatives of the enantiomers in an enantiomeric mixture. For example, an ester derivative of a carboxyl group of the compounds to be separated can be prepared. Certain enzymes will selectively hydrolyze only one of the enantiomers in the mixture. Then the resulting enantiomerically pure acid can be separated from the unhydrolyzed ester.

Resolution of enantiomers may also be accomplished by converting the enantiomers in the mixture to diastereomers by reacting of the former and chiral auxiliaries. The resulting diastereomers can then be separated by column chromatography. This technique is especially useful when the compounds to be separated contain a carboxyl, amino or hydroxyl group that will form a salt or covalent bond with the chiral auxiliary. Chirally pure amino acids, organic carboxylic acids or organosulfonic acids are especially useful as chiral auxiliaries. Once the diastereomers have been separated by chromatography, the individual enantiomers can be regenerated. Frequently, the chiral auxiliary can be recovered and reused.

Compounds of this invention may also contain carbon-carbon double bonds or carbon-nitrogen double bonds in the Z or E configuration, in which the term “Z” represents the larger two substituents on the same side of a carbon-carbon or carbon-nitrogen double bond and the term “E” represents the larger two substituents on opposite sides of a carbon-carbon or carbon-nitrogen double bond. The compounds may also exist as an equilibrium mixture of Z or E configurations.

Compounds of this invention which contain —OH, —NH—, or —CO₂H moieties may have attached thereto prodrug-forming moieties. The prodrug-forming moieties are removed by metabolic processes and release the compounds having the freed hydroxyl, amino, or carboxylic acid in vivo. Prodrugs are useful for adjusting such pharmacokinetic properties of the compounds as solubility and/or hydrophobicity, absorption in the gastrointestinal tract, bioavailability, tissue penetration, and rate of clearance.

Compounds of this invention may exist as acid addition salts, basic addition salts, or zwitterions. Salts of the compounds are prepared during their isolation or following their purification. Acid addition salts of the compounds are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, undecanoate, and their equivalent salts of the compounds and prodrugs thereof are contemplated as being embraced by this invention. When the compounds contain carboxylic acids, basic addition salts may be prepared therefrom by reaction with a base such as the hydroxide, carbonate, and bicarbonate of cations such as lithium, sodium, potassium, calcium, and magnesium.

Compounds of this invention may be administered with or without an excipient. Excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof. Excipients for orally administered compounds in solid dosage forms include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodiumphosphate salts, soybean oil, sucrose, tetrahydrofurfuryl alcohol, and mixtures thereof. Excipients for ophthalmically and orally administered compounds in liquid dosage forms include benzyl alcohol, benzyl benzoate, 1,3-butylene glycol, castor oil, corn oil, cottonseed oil, ethanol, ethyl acetate, ethyl carbonate, fatty acid esters of sorbitan, germ oil, groundnut oil, glycerol, isopropanol, olive oil, polyethylene glycols, propylene glycol, sesame oil, tetrahydrofurfuryl alcohol, water, and mixtures thereof. Excipients for osmotically administered compounds include chlorofluorohydrocarbons, ethanol, isopropanol, water, and mixtures thereof. Excipients for parenterally administered compounds include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for rectally and vaginally administered compounds include cocoa butter, polyethylene glycol, wax, and mixtures thereof.

Compounds of this invention may be administered orally, ophthalmically, osmotically, parenterally (subcutaneously, intramuscularly, intrasternally, intravenously), rectally, topically, transdermally, and vaginally. Orally administered compounds in solid dosage forms may be administered as capsules, dragees, granules, pills, powders, and tablets. Ophthalmically and orally administered compounds in liquid dosage forms may be administered as elixirs, emulsions, microemulsions, solutions, suspensions, and syrups. Osmotically and topically administered compounds may be administered as creams, gels, inhalants, lotions, ointments, pastes, powders, solutions, and sprays. Parenterally administered compounds may be administered as aqueous or oleaginous solutions or aqueous or oleaginous and suspensions, in which suspensions comprise crystalline, amorphous, or otherwise insoluble forms of the compounds. Rectally and vaginally administered compounds may be administered as creams, gels, lotions, ointments, and pastes.

Therapeutically effective amounts of compounds of this invention depend on the recepient of treatment, the disorder being treated and the severity thereof, the composition comprising the compounds, the time of administration, the route of administration, the duration of treatment, the potency of the compounds, and the rate of excretion of the compounds. The daily therapeutically effective amount of the compounds administered to a patient in single or divided doses range from about 0.1 to about 200 mg/kg body weight, preferably from about 0.25 to about 100 mg/kg body weight. Single dose compositions contain these amounts of the compounds or combinations of submultiples thereof.

To determine antibacterial activity of compounds of this invention, twelve petri dishes, each containing successive aqueous dilutions of test compounds in sterilized Brain Heart Infusion agar (Difco 0418-01-5) (10 mL), were inoculated with 1:100 dilutions of the representative microorganisms in TABLE 1 using a Steers replicator block (or 1:10 dilutions for slow-growing Streptococcus strains), co-incubated at 35-37° C. for 20-24 hours with a plate with a control plate having no compound, and inspected visually to provide the minimum inhibitory concentration (MIC), in μg/mL, by which is meant the lowest concentration of the test compound which yielded no growth, a slight haze, or sparsely isolated colonies on the inoculums spot as compared to growth in the control plate.

TABLE 1

| Microorganism | Code |
|---|---|
| Staphylococcus aureus NCTC10G49M | AA |
| Staphylococcus aureus A5177 | BB |
| Staphylococcus aureus PIU 2043 | CC |
| Staphylococcus aureus 1775 | DD |
| Streptococcus pyrogenes EES61 | EE |
| Streptococcus pyrogenes 930 | FF |
| Streptococcus pyrogenes PIU 2548 | GG |
| Streptococcus pneumoniae ATCC 6303 | HH |
| Streptococcus pneumoniae 5979 | JJ |
| Streptococcus pneumoniae 5649 | KK |
| Enterococcus faecalis PIU 1967 | LL |
| Enterococcus faecium GYR 1632 | MM |
| Moraxella catarrhalis 2604 | NN |
| Haemophilus influenzae GYR 1435 | PP |
| Escherichia coil JUHL | QQ |

Compounds of this invention displayed antibacterial activity in the range of about 0.008 μg/mL to greater than about 128 μg/mL against the microorganisms listed in Table 1. This antibacterial activity demonstrates the usefulness of the compounds as antibacterials.

It is meant to be understood that certain metabolites of compounds of this invention, which metabolites are produced by in vitro or in vivo metabolic processes, would also be useful as antibacterials and are meant to be embraced by this invention.

It is also meant to be understood that certain precursor compounds, which precursor compounds may be metabolized in vitro or in vivo to form compounds of this invention, are meant to be embraced by this invention.

Compounds of this invention may also be prepared by synthetic chemical processes, examples of the synthetic chemical processes, and intermediates employed in the processes, are shown hereinbelow. It is meant to be understood that the order of the steps in the processes may be varied, equivalent reagents, solvents, and reaction conditions may be substituted for those specifically mentioned, and vulnerable moieties may be protected and deprotected during the process.

Abbreviations used herein are DME for 1,2-dimethoxyethane; DMF for N,N-dimethylformamide; and THF for tetrahydrofuran. A particular carbonate resin employed in the synthesis of compounds of this invention is MP-carbonate (Argonaut Technologies, San Carlos, Calif.).

Compounds having formula (1) may be prepared as described in commonly-owned U.S. Pat. Nos. 5,866,549, 6,054,435, and 6,075,133.

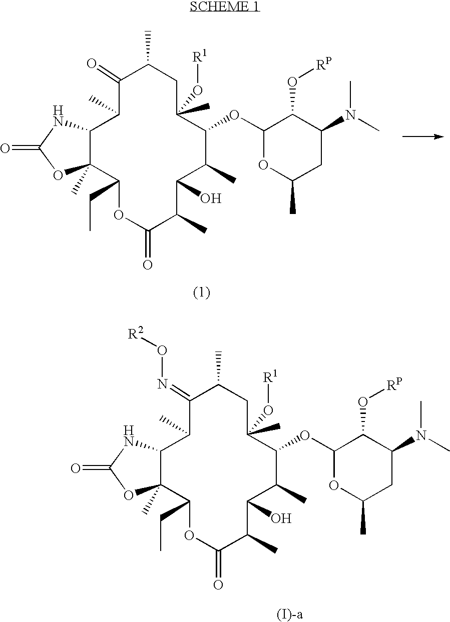

Compounds having formula (1) may be converted to compounds having formula (I)-a by reacting the former, compounds having formula (2)

H₂N—OR² (2),

or salts thereof, with or without, an additive. Additives include camphorsulfonic acid, cesium fluoride, diisopropylethylamine, triethylamine, sodium acetate, boron trifluoride etherate, and carbonate resins. The reaction is typically conducted at about 40° C. to 120° C., over about 1 hour to about 14 days, at pressures of about 1 atm to 5 atm, in solvents such as methanol, ethanol, iso-propanol, dichloromethane, 1,2-dichloroethane, 1,4-dioxane, and DME.

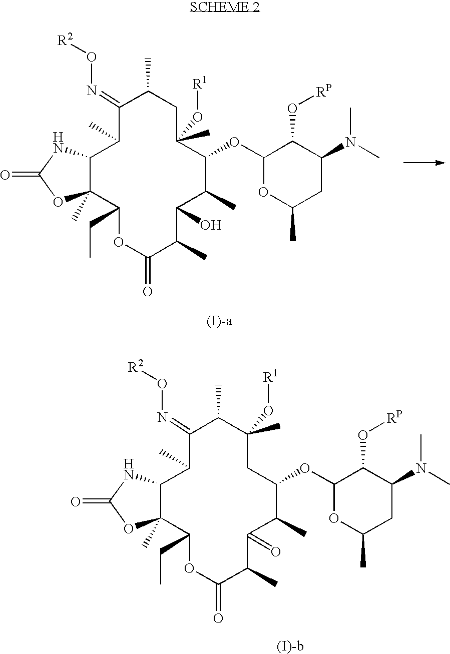

Compounds having formula (I)-a may be converted to compounds having formula (I)-b by reacting the former and an oxidant, with or without a first base. Oxidants include N-chlorosuccinimide.dimethyl sulfide, dicyclohexyl carbodiimide.dimethylsulfoxide with pyridinium trifluoroacetate, and Dess-Martin periodinane. First bases include diisopropylamine, and triethylamine. The reaction is typically conducted at about −20° C. to about 0° C., over about 1 hour to about 2 hours, in solvents such as dichloromethane, chloroform, and 1,2-dichloroethane.

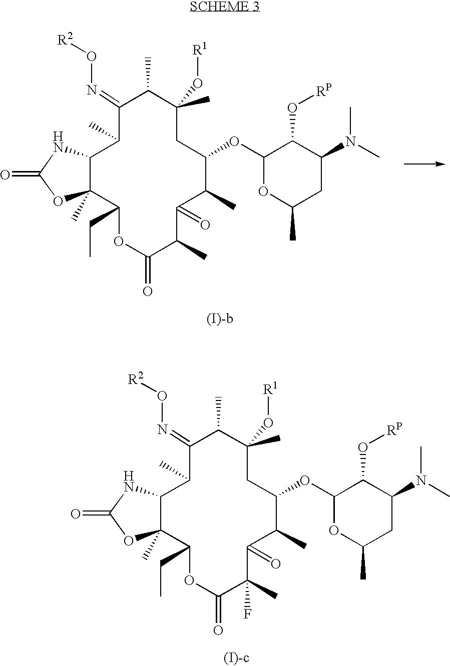

Compounds having formula (I)-b may be converted to compounds having formula (I)-c by reacting the former, a fluoridating agent, and, optionally, a second base. Fluorinating agents include 3,5-dichloro-1-fluoropyridinium tetrafluoroborate, N-fluorobenzenesulfonimide, 3,5-dichloro-1-fluoropyridinium triflate, N-fluoro-N-methyl-para-toluenesulfonamide, N-fluoropyridinium triflate, and N-fluoroperfluoropiperidine. Second bases include sodium hydride, potassium hydride, lithium diisopropylamide, triethylamine, N,N-diisopropylethylamine, lithium bis(trimethylsilyl)amide, sodium bis(trimethylsilyl)amide, and potassium bis(trimethylsilyl)amide. The reactions are typically conducted at about −78° C. to about 0° C., over about 2 to about 24 hours, in solvents such as DMF, THF, and diethyl ether.

Compounds in which R³ is R^{P} and R^{P} is acetyl or benzoyl may be converted to compounds in which R³ is hydrogen by reacting the former and methanol. The reaction is typically conducted at about 25° C. to about 65° C., over about 2 to about 60 hours, in methanol.

Compounds in which R³ is R^{P} and R^{P} is trimethylsilyl may be converted to compounds in which R³ is hydrogen by reacting the former and a fluoride-donating agent. Fluoride-donating agents include tetrabutylammonium fluoride, polymer-bound ammonium fluoride, tetrabutylammonium fluoride, pyridine.hydrogen fluoride, and triethylamine.trihydrofluoride. The reaction is typically conducted at about 0° C. to about 50° C., over about 1 hour to about 24 hours, in solvents such as THF, and 1,4-dioxane.

The compounds and processes of the present invention will be better understood in connection with the following examples.

### EXAMPLE 1

A solution of (2S,3R,4S,6R)-4-(dimethylamino)-2-(((3aS,4R,7R,8S,9S,10R,11S,13R,15R,15aR)-4-ethyl-11-(prop-2-ynyloxy)-8-hydroxy-3a,7,9,11,13,15-hexamethyl-2,6,14-trioxotetradecahydro-2H-oxacyclotetradecino[4,3-d][1,3]oxazol-10-yl)oxy)-6-methyltetrahydro-2H-pyran-3-yl benzoate (1 g), methoxylamine hydrochloride (1.69 g) and boron trifluoride etherate (0.34 mL) in ethanol (10 mL) at 99° C. was stirred in a sealed tube for 14 days then cooled, diluted with isopropyl acetate (100 mL), quenched with 5% sodium bicarbonate (25 mL) and water (100 mL), concentrated to remove the organic solvents, and extracted with isopropyl acetate. The extract was washed with water and brine, dried over anhydrous Na₂SO₄, filtered, and concentrated; and the concentrate was flash chromatographed on silica gel with 25% acetone/hexane.

### EXAMPLE 2

A solution of N-chlorosuccinimide (90 mg) in dichloromethane (2.5 mL) at −10° C. was treated with dimethylsulfide (56 μL), stirred for 10 minutes, treated with a solution of EXAMPLE 1 (350 mg) in dichloromethane (2 mL), stirred at −10° C. to 0° C. for 1.5 hours, treated with triethylamine (63 μL) warmed to room temperature, and washed with saturated sodium carbonate and water. The combined washings were extracted with dichloromethane, and the organics were combined, washed with water and brine, dried over anhydrous Na₂SO₄, filtered, and concentrated.

### EXAMPLE 3

A solution of EXAMPLE 2 (313 mg) in methanol (20 mL) at 55° C. was stirred for 18 hours and concentrated; and the concentrate was flash chromatographed on silica gel with 1-3% 2M NH₃ in methanol/dichloromethane.

### EXAMPLE 4

A solution of EXAMPLE 3 (38.7 mg), 3-bromoquinoline (11.8 μL), triethylamine (5 mL) dichlorobis(triphenylphosphine)palladium(II) (2 mg) and copper iodide (0.1 mg) in acetonitrile (0.5 mL) was stirred at 80° C. for 18 hours then cooled and concentrated; and the concentrate was flash chromatographed on silica gel with 2% 2M NH₃ in methanol/dichloromethane.

### EXAMPLE 5

This example was prepared by substituting 3-bromo-1,5-napthiridine for 3-bromoquinoline in EXAMPLE 4.

### EXAMPLE 6

This example was prepared by substituting 6-bromoquinoxaline for 3-bromoquinoline in EXAMPLE 4.

### EXAMPLE 7

This example was prepared by substituting 2-bromo-5-(3-methyltetrazole)thiophene for 3-bromoquinoline in EXAMPLE 4.

### EXAMPLE 8

This example was prepared by substituting 2-bromo-5-(2-pyridine)thiophene for 3-bromoquinoline in EXAMPLE 4.

### EXAMPLE 9

A solution of EXAMPLE 1 (781 mg) in DMF (5 mL) at −10° C. was treated with 60% oily sodium hydride (81 mg), stirred at −10° C. for 45 minutes, treated with N-fluorobenzenesulfonimide (352 mg), stirred at −10° C. for 3 hours, diluted with iso-propyl acetate, washed with water and brine, and dried (Na₂SO₄), filtered, and concentrated; and the concentrate was flash chromatographed on silica gel with 1.5% 2M NH₃ in methanol/dichloromethane.

### EXAMPLE 10

A solution of Example 9 (630 mg) in methanol (20 mL) at 55° C. was stirred for 18 hours and concentrated; and the concentrate was flash chromatographed on silica gel with 4% 2M NH₃ in methanol/dichloromethane.

### EXAMPLE 11

A solution of EXAMPLE 10 (120 mg), 3-bromoquinoline (35 μL), triethylamine (1.5 mL), dichlorobis(triphenylphosphine)palladium(II) (6.2 mg) and copper iodide (0.3 mg) in acetonitrile (1.5 mL) at 80° C. was stirred for 18 hours then cooled and concentrated; and the concentrate was flash chromatographed on silica gel with 4% 2M NH₃ in methanol/dichloromethane.

### EXAMPLE 12

This example was prepared by substituting 2-bromo-5-(3-methyltetrazole)thiophene for 3-bromoquinoline in EXAMPLE 11.

### SPECTRAL DATA

### EXAMPLE 1

¹³C NMR (CDCl₃) δ 174.5, 167.6, 165.3, 158.8, 132.6, 130.6, 129.7, 128.2, 99.6, 84.0, 80.5, 80.4, 79.9, 77.5, 75.5, 73.5, 72.1, 69.0, 63.3, 61.4, 59.9, 51.1, 43.9, 40.8, 36.8, 36.0, 32.5, 31.9, 25.7, 22.4, 21.1, 19.3, 18.8, 16.6, 15.2, 13.2, 10.3, 7.6.

### EXAMPLE 2

¹³C (CDCl₃) δ 205.4, 169.0, 166.9, 165.1, 158.3, 132.7, 130.4, 129.6, 128.2, 100.8, 83.7, 80.1, 79.5, 76.8, 75.7, 73.5, 71.9, 69.1, 63.5, 61.3, 59.8, 50.8, 50.7, 45.9, 40.7, 37.1, 32.5, 31.5, 25.7, 22.6, 21.0, 19.7, 18.6, 16.5, 14.4, 14.0, 13.4, 10.5.

### EXAMPLE 3

¹³C (CDCl₃) δ 205.4, 169.0, 166.9, 158.3, 103.0, 83.8, 80.2, 79.4, 77.1, 76.6, 73.5, 70.2, 69.4, 65.9, 61.3, 59.8, 50.9, 50.8, 46.5, 40.2, 37.4, 32.5, 28.3, 25.7, 22.6, 21.2, 19.9, 18.7, 16.5, 14.3(2), 13.4, 10.6.

### EXAMPLE 4

¹³C (CDCl₃) δ 205.5, 169.4, 167.8, 158.3, 152.3, 146.8, 138.6, 129.8, 129.2, 127.7, 127.2, 127.0, 117.2, 102.8, 89.3, 83.7, 82.5, 79.7, 77.4, 76.8, 70.2, 69.4, 65.8, 61.5, 60.2, 51.6, 51.0, 46.3, 40.2, 37.6, 32.5, 28.3, 25.8, 22.7, 21.0, 19.5, 18.6, 16.4, 14.5, 13.9, 13.4, 10.6.

### EXAMPLE 5

¹³C (CDCl₃) δ 205.5, 169.6, 167.9, 158.3, 153.1, 151.6, 142.9, 142.7, 139.5, 137.1, 124.4, 120.8, 102.7, 91.2, 83.6, 81.8, 79.8, 77.2, 76.9, 70.1, 69.3, 65.7, 61.5, 60.2, 51.6, 50.9, 46.2, 40.2, 37.6, 32.5, 28.3, 25.8, 22.5, 21.0, 19.4, 18.5, 16.4, 14.5, 14.0, 13.3, 10.6.

### EXAMPLE 6

¹³C (CDCl₃) δ 205.6, 169.5, 167.7, 158.2, 145.4, 144.9, 142.7, 142.5, 132.9, 132.7, 129.3, 125.1, 102.7, 89.1, 84.1, 83.6, 79.6, 77.3, 76.8, 70.1, 69.3, 65.7, 61.5, 60.2, 51.6, 50.9, 46.3, 40.1, 37.5, 32.5, 28.3, 25.8, 22.6, 21.0, 19.5, 18.5, 16.4, 14.5, 14.0, 13.3, 10.6.

### EXAMPLE 7

¹³C (CDCl₃) δ 205.5, 169.2, 167.5, 160.7, 158.1, 133.0, 129.7,-127.4, 125.5, 102.9, 91.7, 83.6, 79.5, 78.0, 77.4, 76.7, 70.2, 69.5, 65.8, 61.5, 60.3, 51.6, 50.9, 46.4, 40.1, 39.4, 37.5, 32.5, 28.2, 25.8, 22.7, 21.1, 19.7, 18.6, 16.4, 14.4, 14.1, 13.4, 10.7.

### EXAMPLE 8

¹³C (CDCl₃) δ 205.5, 169.2, 167.3, 158.1, 152.0, 149.5, 145.6, 136.5, 133.4, 124.7, 124.3, 122.0, 118.8, 102.9, 91.0, 83.6, 79.4, 78.7, 77.4, 76.7, 70.2, 69.4, 65.8, 61.5, 60.3, 51.7, 51.0, 46.3, 40.2, 37.5, 32.5, 28.3, 25.8, 22.7, 21.1, 19.8, 18.6, 16.5, 14.4, 14.1, 13.4, 10.7.

### EXAMPLE 9

¹³C (CDCl₃) δ 203.5, 166.1, 165.5, 165.0, 158.0, 132.8, 130.3, 129.7, 128.3, 101.6, 98.8, 83.6, 80.9, 80.2, 78.5, 78.4, 73.1, 72.0, 69.2, 63.4, 61.3, 59.7, 50.1, 40.7, 37.5, 32.4, 31.3, 28.1, 26.0, 24.7, 22.3, 21.0, 20.5, 18.7, 16.5, 14.7, 13.3, 10.6.

### EXAMPLE 10

¹³C (CDCl₃) δ 204.1, 166.1, 165.6, 158.0, 104.0, 97.9, 83.7, 81.0, 80.2, 79.8, 78.4, 73.1, 70.3, 69.5, 65.8, 61.3, 59.8, 50.1, 40.8, 40.2, 37.7, 32.5, 28.2, 26.1, 25.0, 22.4, 21.1, 20.6, 18.8, 16.6, 15.1, 13.5, 10.6.

### EXAMPLE 11

¹³C (CDCl₃) δ 204.1, 167.3, 165.8, 157.9, 152.4, 146.8, 138.8, 130.0, 129.2, 127.9, 127.2, 127.1, 117.2, 103.9, 98.1, 89.7, 83.6, 82.2, 80.3, 79.6, 78.7, 70.3, 69.5, 65.8, 61.5, 60.3, 51.1, 41.1, 40.2, 38.1, 32.5, 28.3, 26.2, 24.8, 22.4, 21.1, 20.5, 18.7, 16.5, 15.2, 13.4, 10.7.

### EXAMPLE 12

¹³C (CDCl₃) δ 204.2, 167.0, 165.7, 160.7, 158.7, 133.1, 129.9, 127.5, 125.5, 103.9, 98.0, 92.3, 83.5, 80.2, 79.6, 78.7, 77.7, 70.3, 69.5, 65.8, 61.5, 60.4, 51.1, 41.0, 40.2, 39.5, 37.9, 32.5, 28.2, 26.1, 24.9, 22.4, 21.1, 20.7, 18.7, 16.5, 15.1, 13.4, 10.8.

The foregoing is merely illustrative of the invention and is not intended to limit the same to the disclosed compounds and proceses. Variations and changes which are obvious to one skilled in the art are intended to be within the scope and nature of the invention as defined in the claims.

What is claimed is:

1. A compound, or a salt, prodrug, or salt of a prodrug thereof, having formula (I) 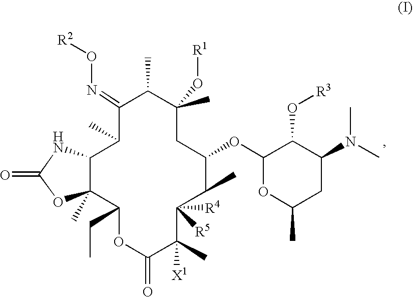
in which
R is alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, and heterocyclyl, —(CH₂)alkenyl substituted with one or two or three substituents independently selected from the group consisting of cycloalkyl, halo, aryl, heteroaryl, and heterocyclyl, or —(CH₂)alkynyl substituted with one or two or three substituents independently selected from the group consisting of cycloalkyl, halo, aryl, heteroaryl, and heterocyclyl;
R² is hydrogen, alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, heterocyclyl, —OH, —OR⁷, ═O, —NH₂, —NHR⁸, and NR⁸R⁹, or —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, or —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;
R³ is hydrogen or R^{P}, in which R^{P} is a hydroxyl protecting moiety;
one of R⁴ and R⁵ is hydrogen and the other is —OH; or R⁴ and R⁵ together are ═O;
R⁶ is alkyl interrupted with one or two or three moieties independently selected from the group consisting of —O—, —NH—, —N(alkyl)-, —S—, —S(O)—, and —SO₂—, or alkyl interrupted with one or two or three moieties independently selected from the group consisting of —O—, —NH—, —N(alkyl)-, —S—, —S(O)—, and —SO₂— and substituted with one or two or three substituents independently selected from the group consisting of cycloalkyl, halo, aryl, heteroaryl, heterocyclyl, —OH, ═O, —OR⁷, —NH₂, —NHR⁸, and —NR⁸R⁹;
R⁷ is alkyl, heteroalkyl, aryl, heteroaryl, heterocyclyl, or alkyl substituted with one substituent selected from the group consisting of aryl, heteroaryl, and heterocyclyl;
R⁸ and R⁹ are independently alkyl, cycloalkyl, heteroalkyl, aryl, heteroaryl, heterocyclyl, or alkyl substituted with one substituent selected from the group consisting of aryl, heteroaryl, and heterocyclyl; or
R⁸ and R⁹ together are C₃-C₇-alkylene or C₄-C₇-alkylene interrupted with one moiety selected from the group consisting of an —O—, —NH—, —N(alkyl)-, —S—, —C(O)—, —S(O)—, and —SO₂—; and
X¹ is hydrogen or fluoro.

2. The compound of claim 1, or a salt, prodrug, or salt of a prodrug thereof, having formula (I), in which
R¹ is —(CH₂)alkenyl, —(CH₂)alkynyl, —(CH₂)alkenyl substituted with one substituent selected from the group consisting of cycloalkyl, halo, aryl, heteroaryl, and heterocyclyl, or —(CH₂)alkynyl substituted with one substituent selected from the group consisting of cycloalkyl, halo, aryl, heteroaryl, and heterocyclyl;
R² is hydrogen, alkyl, —(CH₂)alkenyl, —(CH₂)alkynyl, alkyl substituted with one or two or three substituents independently selected from the group consisting of cycloalkyl, halo, aryl, heteroaryl, heterocyclyl, —OH, —OR ═O, —NH₂, —NHR⁸, and —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, or —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;
R³ is hydrogen or R^{P}, in which R^{P} is a hydroxyl protecting moiety;
one of R⁴ and R⁵ is hydrogen and the other is —OH; or R⁴ and R⁵ together are ═O;
R⁷ is alkyl, heteroalkyl, aryl, heteroaryl, heterocyclyl, or alkyl substituted with one substituent selected from the group consisting of aryl, heteroaryl, and heterocyclyl;
R⁸ and R⁹ are independently alkyl, cycloalkyl, heteroalkyl, aryl, heteroaryl, heterocyclyl, or alkyl substituted with one substituent selected from the group consisting of aryl, heteroaryl, and heterocyclyl; and
X¹ is hydrogen or fluoro.

3. The compound of claim 2, or a salt, prodrug, or salt of a prodrug thereof, having formula (I), in which
R¹ is —(CH₂)alkynyl or —(CH₂)alkynyl substituted with one substituent selected from the group consisting of cycloalkyl, halo, aryl, heteroaryl, and heterocyclyl;
R² is hydrogen, alkyl, —(CH₂)alkenyl, —(CH₂)alkynyl, alkyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, or —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;
R³ is hydrogen or R^{P}, in which R^{P} is a hydroxyl protecting moiety;
one of R⁴ and R⁵ is hydrogen and the other is —OH; or R⁴ and R⁵ together are ═O;
R⁷ is alkyl, heteroalkyl, aryl, heteroaryl, heterocyclyl, or alkyl substituted with one substituent selected from the group consisting of aryl, heteroaryl, and heterocyclyl;
R⁸ and R⁹ are independently alkyl, cycloalkyl, heteroalkyl, aryl, heteroaryl, heterocyclyl, or alkyl substituted with one substituent selected from the group consisting of aryl, heteroaryl, and heterocyclyl; and
X¹ is hydrogen or fluoro.

4. The compound of claim 3, or a salt, prodrug, or salt of a prodrug thereof, having formula (I), in which
R¹ is —(CH₂)alkynyl or —(CH₂)alkynyl substituted with one substituent selected from the group consisting of aryl and heteroaryl;
R² is hydrogen, alkyl, or alkyl substituted with one substituent selected from the group consisting of cycloalkyl, halo, aryl, heteroaryl, and heterocyclyl;
R³ is hydrogen or R^{P}, in which R^{P} is a hydroxyl protecting moiety;
one of R⁴ and R⁵ is hydrogen and the other is —OH; or R⁴ and R⁵ together are ═O; and
X¹ is hydrogen or fluoro.

5. The compound of claim 4, or a salt, prodrug, or salt of a prodrug thereof, having formula (I), in which
R¹ is —(CH₂)alkynyl or —(CH₂)alkynyl substituted with one substituent selected from the group consisting of aryl and heteroaryl;
R² is hydrogen, alkyl, or alkyl substituted with phenyl;
R³ is hydrogen or R^{P}, in which R^{P} is a hydroxyl protecting moiety;
one of R⁴ and R⁵ is hydrogen and the other is —OH; or R⁴ and R⁵ together are ═O; and
X¹ is hydrogen or fluoro.

6. The compound of claim 5, or a salt, prodrug, or salt of a prodrug thereof, having formula (I), in which
R¹ is —(CH₂)alkynyl or —(CH₂)alkynyl substituted with one substituent selected from the group consisting of aryl and heteroaryl;
R² is alkyl;
R³ is hydrogen or R^{P}, in which R^{P} is a hydroxyl protecting moiety;
one of R⁴ and R⁵ is hydrogen and the other is —OH; or R⁴ and R⁵ together are ═O; and
X¹ is hydrogen or fluoro.

7. The compound of claim 6, or a salt, prodrug, or salt of a prodrug thereof, having formula (I), in which
R¹ is —(CH₂)alkynyl or —(CH₂)alkynyl substituted with one substituent selected from the group consisting of quinoxalinyl, naphthyridinyl, quinolinyl, isoquinolinyl, thienyl, isoxazolyl, thiazolyl, pyridyl and pyrimidinyl, in which the thienyl, isoxazolyl, thiazolyl, pyridyl and pyrimidinyl are independently unsubstituted or substituted with one substituent selected from the group consisting of pyrimidinyl, pyridyl, thienyl, isoxazolyl, thiazolyl, 1H-tetraäzol-5-yl, 2H-tetraäzol-5-yl, 1-methyl-1H-tetraäzol-5-yl, and 2-methyl-2H-tetraäzol-5-yl;
R² is alkyl;
R³ is hydrogen or R^{P}, in which R^{P} is a hydroxyl protecting moiety;
one of R⁴ and R⁵ is hydrogen and the other is —OH; or R⁴ and R⁵ together are ═O; and
X¹ is hydrogen or fluoro.

8. The compound of claim 7, or a salt, prodrug, or salt of a prodrug thereof, having formula (I), in which
R¹ is prop-2-ynyl, 3-(quinolin-3-yl)prop-2-ynyl, 3-(1,5-naphthyridin-3-yl)prop-2-ynyl, 3-(quinoxalin-6-yl)prop-2-ynyl, 3-(5-(2-methyl-2H-tetraäzol-5-yl)thien-2-yl)prop-2-ynyl, 3-(5-(pyridin-2-yl)thien-2-yl)prop-2-ynyl, 3-(2-(pyrimidin-2-yl)thien-5-yl)prop-2-ynyl, 3-(2-(2-methyl-2H-tetraäzol-5-yl)1,3-thiazol-5-yl)prop-2-ynyl, 3-(2-(2-methyl-2H-tetraäzol-5-yl)pyridin-6-yl)prop-2-ynyl, 3-(3-(pyridin-2-yl)isoxazol-5-yl)prop-2-ynyl, or 3-(3-(2-methyl-2H-tetraäzol-5-yl)isoxazol-5-yl)prop-2-ynyl;
R² is alkyl;
R³ is hydrogen or R^{P}, in which R^{P} is a hydroxyl protecting moiety;
one of R⁴ and R⁵ are hydrogen and the other is —OH; or R⁴ and R⁵ together are ═O; and
X¹ is hydrogen or fluoro.

9. The compound of claim 6, or a salt, prodrug, or salt of a prodrug thereof, having formula (I), in which
R¹ is —(CH₂)alkynyl or —(CH₂)alkynyl substituted with one substituent selected from the group consisting of aryl and heteroaryl;
R² is methyl;
R³ is hydrogen or R^{P}, in which R^{P} is a hydroxyl protecting moiety;
one of R⁴ and R⁵ is hydrogen and the other is —OH; or R⁴ and R⁵ together are —O; and
X¹ is hydrogen or fluoro.

10. The compound of claim 1, or a salt, prodrug, or salt of a prodrug thereof, having formula (I) 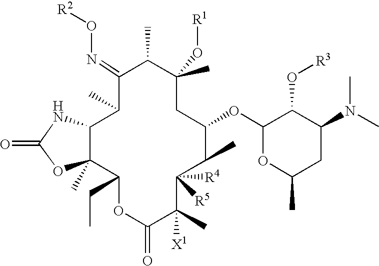
in which R¹ is —(CH₂)alkynyl substituted with one substituent selected from the group consisting of naphthyridinyl, quinolinyl, quinoxalinyl, and thienyl, in which the thienyl is substituted with one substituent selected from the group consisting of pyridyl and 2H-tetraäzolyl, in which the 2H-tetraäzolyl is substituted with alkyl; R² is alkyl; R³ is hydrogen; R⁴ and R⁵ taken together are ═O; and X¹ is hydrogen or fluoro.

11. The compound of claim 1, or a salt, prodrug, or salt of a prodrug thereof, having formula (I) 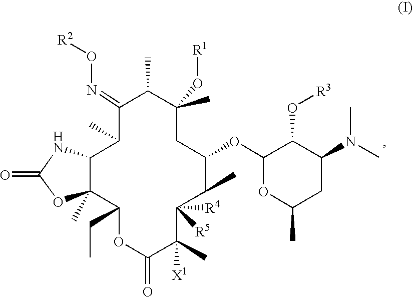
in which R¹ is —(CH₂)—C₂-alkynyl substituted with one substituent selected from the group consisting of naphthyridinyl, quinolinyl, quinoxalinyl, and thienyl, in which the thienyl is substituted with one substituent selected from the group consisting of pyridyl and 2H-tetraäzolyl, in which the 2H-tetraäolyl is substituted with C₁-alkyl; R² is C₁-alkyl; R³ is hydrogen; R⁴ and R⁵ taken together are ═O; and X¹ is hydrogen or fluoro.

12. The compound of claim 11, or salt, prodrug, or salt of a prodrug, in which R¹ is 3-(quinolin-3-yl)prop-2-ynyl, 3-(1,5-naphthyridin-3-yl)prop-2-ynyl, or 3-(quinoxalin-6-yl)prop-2-ynyl; R² is methyl; R³ is hydrogen; R⁴ and R⁵ taken together are ═O; and X¹ is hydrogen.

13. The compound of claim 11, or salt, prodrug, or salt of a prodrug, in which R¹ is 3-(quinolin-3-yl)prop-2-ynyl, 3-(1,5-naphthyridin-3-yl)prop-2-ynyl, or 3-(quinoxalin-6-yl)prop-2-ynyl; R² is methyl; R³ is hydrogen; R⁴ and R⁵ taken together are ═O; and X¹ is fluoro.

14. The compound of claim 11, or salt, prodrug, or salt of a prodrug, in which R¹ is 3-(5-(2-methyl-2H-tetraäzol-5-yl)thien-2-yl)prop-2-ynyl, or 3-(5-(pyridin-2-yl)thien-2-yl)prop-2-ynyl; R² is methyl; R³ is hydrogen; R⁴ and R⁵ taken together are ═O; and X¹ is hydrogen.

15. The compound of claim 11, or salt, prodrug, or salt of a prodrug, in which R¹ is 3-(5-(2-methyl-2H-tetraäzol-5-yl)thien-2-yl)prop-2-ynyl, or 3-(5-(pyridin-2-yl)thien-2-yl)prop-2-ynyl; R² is methyl; R³ is hydrogen; R⁴ and R⁵ taken together are ═O; and X¹ is fluoro.

16. A composition for the treatment or prophylaxis of bacterial infections in a fish or a mammal comprising a therapeutically effective amount of a compound of claim 1 and an excipient.

17. A method for prophylaxis or treatment of bacterial infections in a fish or a mammal comprising administering to the fish or the mammal a therapeutically effective amount of a compound of claim 1.

18. A compound of claim 1, or a salt, prodrug, or salt of a prodrug thereof, which is
(3aS,4R,7R,9R,10R,11S,13R,14E,15R,15aR)-4-ethyl-14-(methoxyimino)-3a,7,9,11,13,15-hexamethyl-2,6,8-trioxo-11-((3-quinolin-3-ylprop-2-ynyl)oxy)tetradecahydro-2H-oxacyclotetradecino(4,3-d)(1,3)oxazol-10-yl 3,4,6-trideoxy-3-(dimethylamino)-beta-D-xylo-hexopyranoside;
(3aS,4R,7R,9R,10R,11S,13R,14E,15R,15aR)-4-ethyl-14-(methoxyimino)-3a,7,9,11,13,15-hexamethyl-11-((3-(1,5-naphthyridin-3-yl)prop-2-ynyl)oxy)-2,6,8-trioxotetradecahydro-2H-oxacyclotetradecino(4,3-d)(1,3)oxazol-10-yl 3,4,6-trideoxy-3-(dimethylamino)-β-D-xylo-hexopyranoside;
(3aS,4R,7R,9R,10R,11S,13R,14E,15R,15aR)-4-ethyl-14-(methoxyimino)-3a,7,9,11,13,15-hexamethyl-2,6,8-trioxo-11-((3-quinoxalin-6-ylprop-2-ynyl)oxy)tetradecahydro-2H-oxacyclotetradecino(4,3-d)(1,3)oxazol-10-yl 3,4,6-trideoxy-3-(dimethylamino)-β-D-xylo-hexopyranoside;
(3aS,4R,7R,9R,10R,11S,13R,14E,15R,15aR)-4-ethyl-14-(methoxyimino)-3a,7,9,11,13,15-hexamethyl-11-((3-(5-(2-methyl-2H-tetraäzol-5-yl)thien-2-yl)prop-2-ynyl)oxy)-2,6,8-trioxotetradecahydro-2H-oxacyclotetradecino(4,3-d)(1,3)-oxazol-10-yl 3,4,6-trideoxy-3-(dimethylamino)-β-D-xylohexopyranoside;
(3aS,4R,7R,9R,10R,11S,13R,14E,15R,15aR)-4-ethyl-14-(methoxyimino)-3a,7,9,11,13,15-hexamethyl-2,6,8-trioxo-11-((3-(5-pyridin-2-ylthien-2-yl)prop-2-ynyl)oxy)tetradecahydro-2H-oxacyclotetradecino(4,3-d)(1,3)oxazol-10-yl 3,4,6-trideoxy-3-(dimethylamino)-β-D-xylo-hexopyranoside;
(3aS,4R,7S,9R,10R,11S,13R,14E,15R,15aR)-4-ethyl-7-fluoro-14-(methoxyimino)-3a,7,9,11,13,15-hexamethyl-2,6,8-trioxo-11-((3-quinolin-3-ylprop-2-ynyl)oxy)tetradecahydro-2H-oxacyclotetradecino(4,3-d)(1,3) oxazol-10-yl 3,4,6-trideoxy-3-(dimethylamino)-β-D-xylo-hexopyranoside; or
(3aS,4R,7S,9R,10R,11S,13R,14E,15R,15aR) -4-ethyl-7-fluoro-14-(methoxyimino)-3a,7,9,11,13,15-hexamethyl-11-((3-(5-(2-methyl-2H-tetraäzol-5-yl)thien-2-yl)prop-2-ynyl)oxy)-2,6,8-trioxotetradecahydro-2H-oxacyclotetradecino-(4,3-d)(1,3)oxazol-10-yl 3,4,6-trideoxy-3-(dimethylamino)-β-D-xylo-hexopyranoside.

19. A processes for making a compound having formula (I)-d 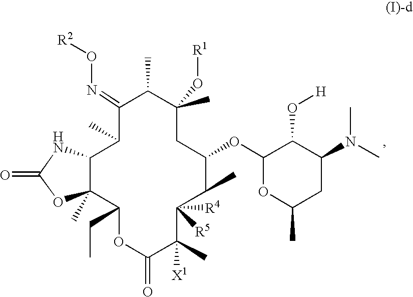
or a salt, prodrug, or salt of a prodrug thereof,
in which
R¹ is alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, and heterocyclyl, —(CH₂)alkenyl substituted with one or two or three substituents independently selected from the group consisting of cycloalkyl, halo, aryl, heteroaryl, and heterocyclyl, or —(CH₂)alkynyl substituted with one or two or three substituents independently selected from the group consisting of cycloalkyl, halo, aryl, heteroaryl, and heterocyclyl;
R² is hydrogen, alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, heterocyclyl —OH, —OR⁷, ═O, —NH₂, —NHR⁸, and —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR, and —NR⁸R⁹, or —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;
R⁴ and R⁵ taken together are ═O;
R⁶ is alkyl interrupted with one or two or three moieties independently selected from the group consisting of —O—, —NH—, —N(alkyl)-, —S—, —S(O)—, and —SO₂— or alkyl interrupted with one or two or three moieties independently selected from the group consisting of —O—, —NH—, —N(alkyl)-, —S—, —S(O)—, and —SO₂— and substituted with one or two or three substituents independently selected from the group consisting of cycloalkyl, halo, aryl, heteroaryl, heterocyclyl —OH, ═O, —OR⁷, —NH₂, —NHR⁸, and —NR⁸R⁹;
R⁷ is alkyl, heteroalkyl, aryl, heteroaryl, heterocyclyl, or alkyl substituted with one substituent selected from the group consisting of aryl, heteroaryl, and heterocyclyl;
R⁸ and R⁹ are independently alkyl, cycloalkyl, heteroalkyl, aryl, heteroaryl, heterocyclyl, or alkyl substituted with one substituent selected from the group consisting of aryl, heteroaryl, and heterocyclyl; or
R⁸ and R⁹ together are C₃-C₇-alkylene or C₄-C₇-alkylene interrupted with one moiety selected from the group consisting of —O—, —NH—, —N(alkyl)-, —S—, —C(O)—, —S(O)—, and —SO₂—; and
X¹ is hydrogen,
the process comprising the steps of:
(a) reacting a compound having formula (1) 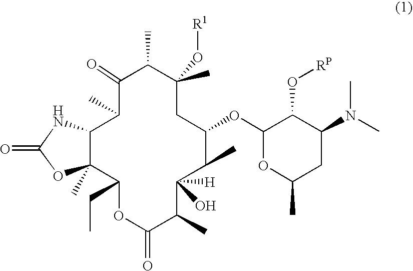
in which R^{P} is acetyl or benzoyl, and a compound having formula (2)
H₂N—OR² (2),
or a salt thereof, with or without an additive, at a pressure between about 1.2 and 5 atm, to provide a compound having formula (I)-a 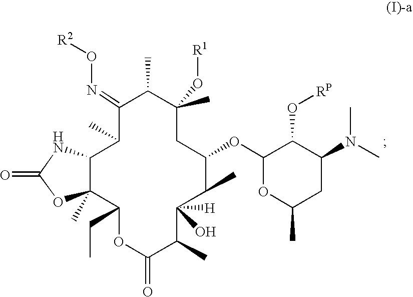
(b) reacting the product of step (a) and an oxidant, with or without a first base, to provide a compound having formula (I)-b 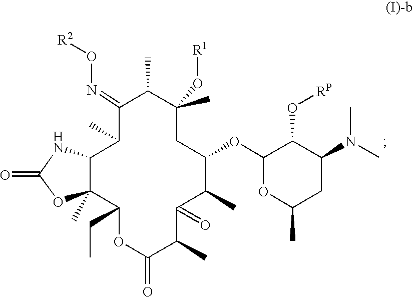
and
(c) reacting the product of step (b) and methanol.

20. A process for making a compound having formula (I)-c 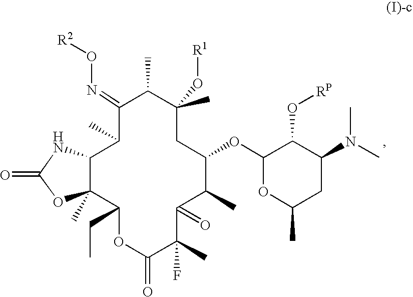
or a salt, prodrug, or salt of a prodrug thereof,
in which
R¹ is alkyl, —(CH₂)alkenyl, —(CH₂)alkynyl, —CH₂R⁶, alkyl substituted with one or two or three substituents independently selected from the group consisting of cycloalkyl, halo, aryl, heteroaryl, and heterocyclyl, —(CH₂)alkenyl substituted with one or two or three substituents independently selected from the group consisting of cycloalkyl, halo, aryl, heteroaryl, and heterocyclyl, or —(CH₂)alkynyl substituted with one or two or three substituents independently selected from the group consisting of cycloalkyl, halo, aryl, heteroaryl, and heterocyclyl;
R² is hydrogen, alkyl, —(CH₂)alkenyl, —(CH₂)alkynyl, —CH₂R⁶, alkyl substituted with one, two, or three substituents independently selected from the group consisting of cycloalkyl, halo, aryl, heteroaryl, heterocyclyl, —OH, —OR⁷, ═O, —NH₂, —NHR⁸, or —NR⁸R⁹, —(CH₂)alkenyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹, and —(CH₂)alkynyl substituted with one or two or three substituents independently selected from the group consisting of cycloalkyl, halo, aryl, heteroaryl, heterocyclyl, —OH, —OR⁷, ═O, —NH₂, —NHR⁸, and —NR⁸R⁹;
R^{P} is acetyl or benzoyl;
R⁶ is alkyl interrupted with one or two or three moieties independently selected from the group consisting of —O—, —NH—, —N(alkyl)-, —S—, —S(O)—, and —SO₂— or alkyl independently interrupted with one or two or three moieties independently selected from the group consisting of —O—, —NH—, —N(alkyl)-, —S—, —S(O)—, and —SO₂— and substituted with one or two or three substituents independently selected from the group consisting of cycloalkyl, halo, aryl, heteroaryl, heterocyclyl —OH, ═O, —OR⁷, —NH₂, —NHR⁸, and —NR⁸R⁹;
R⁷ is alkyl, heteroalkyl, aryl, heteroaryl, heterocyclyl, or alkyl substituted with one substituent selected from the group consisting of aryl, heteroaryl, and heterocyclyl;
R⁸ and R⁹ are independently alkyl, cycloalkyl, heteroalkyl, aryl, heteroaryl, heterocyclyl, or alkyl substituted with one substituent independently selected from the group consisting of aryl, heteroaryl, and heterocyclyl; or
R⁸ and R⁹ together are C₃-C₇-alkylene or C₄-C₇-alkylene interrupted with one moiety selected from the group consisting of —O—, —NH—, —N(alkyl)-, —S—, —C(O)—, —S(O)—, and —SO₂—,
the process comprising the step of:
(a) reacting a compound having formula (I)-b 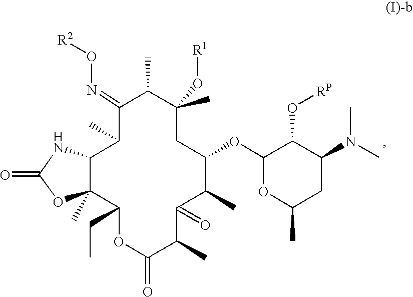
and a fluoridating agent, with or without a second base.
